(19) United States
(12) Patent Application Publication
(10) Pub. No.: US 2012/0157850 A1
(43) Pub. Date: Jun. 21, 2012

(54) ULTRASOUND DIAGNOSIS APPARATUS

(75) Inventors: Atsushi Sumi, Otawara-shi (JP); Kenichi Ichioka, Nasushiobara-shi (JP); Shigemitsu Nakaya, Nasushiobara-shi (JP)

(73) Assignees: Toshiba Medical Systems Corporation, Tochigi (JP); Kabushiki Kaisha Toshiba, Tokyo (JP)

(21) Appl. No.: 13/389,610

(22) PCT Filed: Aug. 30, 2011
(86) PCT No.: PCT/JP2011/004818

§ 371 Date: Feb. 9, 2012

(30) Foreign Application Priority Data
Aug. 30, 2010 (JP) 2010-191877
Aug. 29, 2011 (JP) 2011-186302

(51) Int. Cl. A61B 8/14

(52) U.S. Cl. 600/443

(57) ABSTRACT

An ultrasound diagnostic device that is able to generate images with high sensitivity for structures and unnoticeable artifacts. The ultrasound diagnosis apparatus includes an image-capturing part, a calculating part, and a composition part. The image-capturing part deflects ultrasound waves at a plurality of different deflection angles to transmit ultrasound waves to a subject, receives echo signals from the subject, and generates a plurality of ultrasound image data in which the deflection angles of the ultrasound waves are different in each. The calculating part obtains, based on the plurality of ultrasound image data, the trend of the angular dependence of the plurality of ultrasound image data on the deflection angles. The composition part changes the weights of the plurality of ultrasound image data in accordance with the trend of the angular dependence, and composes the plurality of ultrasound image data.

### TECHNICAL FIELD

Embodiments described herein relate generally to an ultrasound diagnosis apparatus.

### BACKGROUND ART

For an ultrasound diagnosis apparatus, there is technology known as compound scanning. In compound scanning, ultrasound waves (transmission beams) are transmitted to a subject by changing the deflection angle. Furthermore, in compound scanning, a plurality of ultrasound image data is generated based on each transmission beam that has a different deflection angle. Furthermore, in compound scanning, these multiple ultrasound image data are composed and displayed. One example of a method of compounding ultrasound image data is averaging. The strength of echo signals received by the ultrasound diagnosis apparatus changes depending on the angle formed by a structure inside an organism and a transmission beam. For example, if the short axis cross-section of a blood vessel is scanned using a linear ultrasound probe, the intima above and below the blood vessel is visualized relatively clearly, but the intima on the left and right is difficult to visualize. Therefore, by compounding a plurality of ultrasound images in which the deflection angle of the transmission beam has been changed, deficits in echo signals caused by angular dependence are mutually interpolated. As a result, it is possible to more clearly visualize the structure. Moreover, when the deflection angle of the transmission beam changes, the generated position and strength of artifacts such as side lobes or reverberation change. By averaging a plurality of ultrasound images in which the deflection angle of the transmission beam has been changed, it is possible to relatively reduce artifacts.

### PRIOR ART LITERATURE

### Patent Document

[Patent Document 1] Japanese Unexamined Patent Application Publication No. 2009-82469

### OUTLINE OF THE INVENTION

### Problem To Be Solved By the Invention

However, when scanning a certain area, even if image data with high sensitivity is obtained with transmission beams of a specific deflection angle, when scanning is performed with transmission beams of a different deflection angle, image data with reduced sensitivity may be obtained. When image data with high sensitivity and image data with low sensitivity are thus averaged, the signal strength of that area is decreased. Methods of preventing decreases in signal strength include a method of displaying the maximum value among the pixel values of a specific position from among a plurality of ultrasound image data. Based on this method, it is possible to prevent decreases in signals of a structure, but artifacts may become more noticeable.

The objective of the embodiment is to provide an ultrasound diagnosis apparatus that is able to generate an image of a structure in which the sensitivity is high and artifacts are not noticeable.

### Means For Solving the Problem

An ultrasound diagnosis apparatus according the present embodiment, includes an image-capturing part, a calculator and a composition part. The image-capturing part deflects ultrasound waves at a plurality of different deflection angles, transmits ultrasound waves to a subject, receives echo signals from the subject, and generates a plurality of ultrasound image data in which the deflection angles of the ultrasound waves are different for each. The calculator, based on the plurality of ultrasound image data, obtains the trend of the angular dependence of the plurality of ultrasound image data on the deflection angles. The composition part weighs each of the plurality of ultrasound image data in accordance with the trend of said angular dependence, and composes the plurality of ultrasound image data.

### BRIEF DESCRIPTION OF THE DRAWINGS

FIG. 1 is a block diagram of the ultrasound diagnosis apparatus according to the first embodiment.

FIG. 2 is a diagram illustrating the concept of scanning.

FIG. 3 is a diagram schematically showing ultrasound images.

FIG. 4 is a diagram illustrating the concept of scanning.

FIG. 5 is a flowchart showing the series of operations by the ultrasound diagnosis apparatus according to the first embodiment.

FIG. 6 is a block diagram of the ultrasound diagnosis apparatus according to the second embodiment.

FIG. 7 is a flowchart showing the series of operations by the ultrasound diagnosis apparatus according to the second embodiment.

### MODE FOR CARRYING OUT THE INVENTION

### First Embodiment

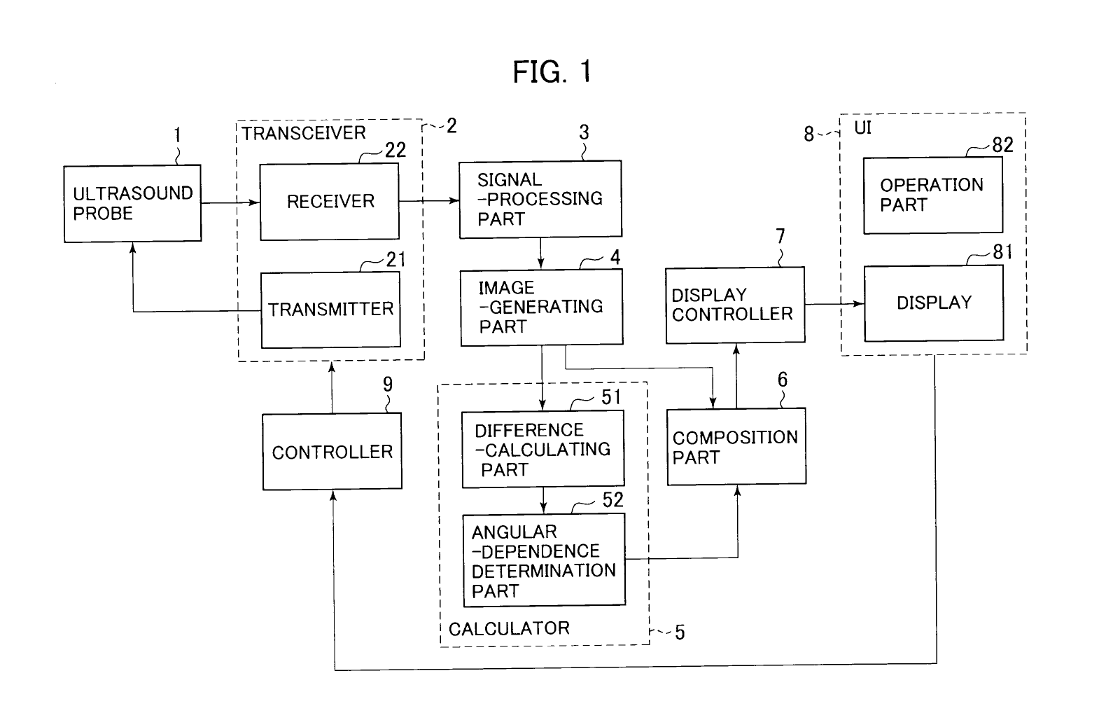

An ultrasound diagnosis apparatus according to a first embodiment is explained with reference to FIG. 1. FIG. 1 is a block diagram of an ultrasound diagnosis apparatus according to the first embodiment. The ultrasound diagnosis apparatus according to the first embodiment includes an ultrasound probe 1, a transceiver 2, a signal-processing part 3, an image-generating part 4, a calculator 5, a composition part 6, a display controller 7, a user interface (UI) 8, and a controller 9.

### Ultrasound Probe 1

For the ultrasound probe 1, a one-dimensional array probe in which multiple ultrasound transducers are arranged in a single row in the scanning direction may be used, or a two-dimensional array probe in which multiple ultrasound transducers are arranged two-dimensionally may be used. The ultrasound probe 1 transmits ultrasound waves to a subject, and receives reflected waves from the subject as echo signals.

### Transceiver 2

The transceiver 2 includes a transmitter 21 and a receiver 22. The transceiver 2 feeds electrical signals to the ultrasound probe 1 to generate ultrasound waves. Moreover, the transceiver 2 receives echo signals received by the ultrasound probe 1.

### Transmitter 21

The transmitter 21 feeds electrical signals to the ultrasound probe 1 to generate ultrasound waves. The transmitter 21 feeds electrical signals to the ultrasound probe 1 to transmit ultrasound waves that are beam-formed (transmission beam-formed) to a prescribed focal point. The transmitter 21 includes, for example, a clock generator, a transmission delay circuit, and a pulser circuit that are not shown in the diagram. The clock generator generates clock signals that determine the transmission timing and transmission frequency of ultrasound signals. The transmission delay circuit applies a delay at the time of transmission of ultrasound waves. Specifically, the transmission delay circuit focuses the ultrasound waves at a prescribed depth based on a delay time for focusing, transmits the ultrasound waves in a prescribed direction based on a delay time for deflection, and performs transmission focus. The pulser circuit includes pulsers for several individual channels corresponding to each ultrasound transducer. The pulser circuit generates a drive pulse at a transmission timing to which a delay has been applied, and feeds the drive pulse to each ultrasound transducer of the ultrasound probe 1.

### Receiver 22

The receiver 22 receives echo signals received by the ultrasound probe 1. Moreover, the receiver 22 performs a delay process for the echo signals. As a result of this process, the analog echo signals are converted into phased (reception beam-formed) digital data. The receiver 22 includes, for example, a preamplifier circuit, an A/D converter, a reception delay circuit, and an adder that are not shown in the diagram. The preamplifier circuit amplifies echo signals output from each ultrasound transducer of the ultrasound probe 1 for each reception channel. The A/D converter converts the amplified echo signals into digital signals. To the echo signals that have been converted into digital signals, the reception delay circuit applies a delay time necessary for determining the reception directivity. Specifically, the reception delay circuit applies to the digital echo signals a focusing delay time for focusing ultrasound waves from a prescribed depth, and a reception delay time for deflection for setting the reception directivity relative to a prescribed direction. The adder adds the echo signals to which the delay time has been applied. As a result of this addition, reflected components from the direction corresponding to the reception directivity are emphasized. In other words, echo signals obtained from a prescribed direction are phased and added by the reception delay circuit and the adder. The receiver 22 outputs the echo signals that have undergone the delay process to the signal-processing part 3.

### Signal-Processing Part 3

The signal-processing part 3 includes a B-mode processing part. The B-mode processing part receives echo signals from the receiver 22 and performs visualization of the amplitude information of the echo signals. Specifically, the B-mode processing part performs a band-pass filtration process on the echo signals. Then, the B-mode processing part detects the envelope curve of the output signals, and performs a compression process based on logarithmic conversion on the detected data. Moreover, the signal-processing part 3 may include a CFM (Color Flow Mapping) processing part. The CFM processing part performs visualization of blood-flow information. Blood-flow information is obtained as binarized information. Blood-flow information includes information such as velocity, distribution, or power. Moreover, the signal-processing part 3 may include a Doppler processing part. The Doppler processing part performs phase detection of the echo signals to extract a Doppler shift frequency component. Moreover, the Doppler processing part generates a Doppler frequency distribution representing the blood-flow velocity by performing an FFT process. The signal-processing part 3 outputs the echo signals (ultrasound raster data) that have undergone signal processing to the image-generating part 4.

### Image-Generating Part 4

The image-generating part 4 generates ultrasound image data based on signal-processed echo signals (ultrasound raster data) output from the signal-processing part 3. The image-generating part 4 includes, for example, a DSC (Digital Scan Converter). The image-generating part 4 converts signal-processed echo signals represented in signal sequences of the scanning lines into image data represented in an orthogonal coordinate system (scan conversion process). The image-generating part 4 performs a scan conversion process on echo signals that have undergone signal processing by the B-mode processing part. As a result of this scan conversion process, B-mode image data representing the shape of a tissue of the subject is generated. The image-generating part 4 outputs ultrasound image data to both the calculator 5 and the composition part 6.

For example, the ultrasound probe 1 and the transceiver 2 scan a cross-section inside the subject using ultrasound waves. The image-generating part 4 generates B-mode image data (tomographic image data) providing a two-dimensional representation of the shape of a tissue in the cross-section. Moreover, the ultrasound probe 1 and the transceiver 2 may also acquire volume data by scanning a three-dimensional region with ultrasound waves. In this case, the image-generating part 4 may perform volume rendering on the volume data. As a result of volume rendering, three-dimensional image data representing the shape of a tissue in three dimensions are generated. Alternatively, the image-generating part 4 may perform an MPR (Multi Planar Reconstruction) process on the volume data. As a result of the MPR process, image data (MPR image data) of an arbitrary cross-section is generated. It should be noted that the ultrasound probe 1, the transceiver 2, the signal-processing part 3, and the image-generating part 4 configure one example of an “image-capturing part.”

The ultrasound diagnosis apparatus according to the present embodiment may include an image memory (not shown). The image memory stores data obtained by the ultrasound diagnosis apparatus according to the present embodiment. For example, the image memory stores echo signals output from the receiver 22. Moreover, the image memory may store ultrasound raster data output from the signal-processing part 3. Moreover, the image memory may store ultrasound image data, such as tomographic image data, output from the image-generating part 4.

### Compound Scanning

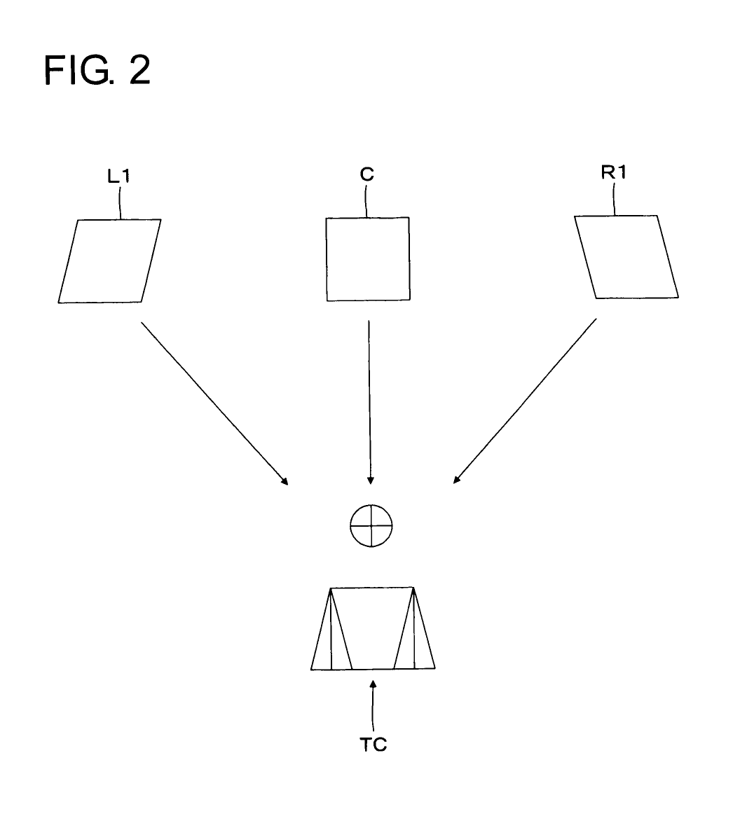

The ultrasound diagnosis apparatus according to the present embodiment deflects ultrasound waves at multiple different deflection angles and transmits and receives ultrasound waves. Moreover, based on the received echo signals, the ultrasound diagnosis apparatus generates a plurality of ultrasound image data in which the deflection angle of the ultrasound waves is different for each. For example, the controller 9 controls the deflection angle. The controller 9 outputs control signals including information indicating a deflection angle to the transceiver 2. The transceiver 2, under the control of the controller 9, changes the deflection angle and transmits and receives ultrasound waves. The operator may input a desired deflection angle using an operation part 82, or the deflection angle may be preset in the controller 9. For example, when the operator inputs multiple different deflection angles using the operation part 82, information indicating each deflection angle is output from the user interface (UI) 8 to the controller 9. The controller 9 controls the transmission and reception of ultrasound waves by the transceiver 2 in accordance with the deflection angles input from the operation part 82. It should be noted that a scan in which ultrasound waves are transmitted and received in the directions of multiple different deflection angles is sometimes referred to as a compound scan. The following is a description of a compound scan, with reference to FIG. 2. FIG. 2 is a diagram showing a concept of a scan. In the present embodiment, a case is described in which tomographic image data is generated as one example of ultrasound image data.

As an example, the following is a description of a case in which ultrasound waves are deflected at three different deflection angles and the ultrasound waves are transmitted and received, and three tomographic image data that each have different deflection angles is generated. Under the control of the controller 9, the transceiver 2 deflects ultrasound waves at each of a first deflection angle, a second deflection angle, and a third deflection angle and transmits and receives ultrasound waves. The first deflection angle is the angle between the second deflection angle and the third deflection angle. The following is a description of a case in which, as an example of the first deflection angle, the angle of deflection is 0°. In other words, the first deflection angle corresponds to an angle in which the ultrasound waves are not deflected. The second deflection angle and the third deflection angle are deflection angles on mutually opposite sides in regard to the first deflection angle. The first deflection angle, the second deflection angle, and the third deflection angle may be input by the operator using the operation part 82.

The image-generating part 4 generates tomographic image data C in which the ultrasound waves have been deflected at the first deflection angle. Moreover, the image-generating part 4 generates tomographic image data L1 in which the ultrasound waves have been deflected at the second deflection angle. Moreover, the image-generating part 4 generates tomographic image data R1 in which the ultrasound waves have been deflected at the third deflection angle. FIG. 2 is a schematic diagram of the respective tomographic image data. The tomographic image data C shown in FIG. 2 is image data in which the ultrasound waves have been deflected at the first deflection angle (deflection angle of 0°). In other words, the tomographic image data C is image data obtained without deflecting the ultrasound waves. The tomographic image data L1 is image data in which the ultrasound waves have been deflected at the second deflection angle (left side in FIG. 2). The tomographic image data R1 is image data in which the ultrasound waves have been deflected at the third deflection angle (right side in FIG. 2). The tomographic image data C, the tomographic image data L1, and the tomographic image data R1 are composed by the composition part 6 described below. As a result of this composition, the composite image data TC shown in FIG. 2 is generated.

### Relationship Between the Deflection Angle of the Ultrasound Waves And the Echo Signal Strength

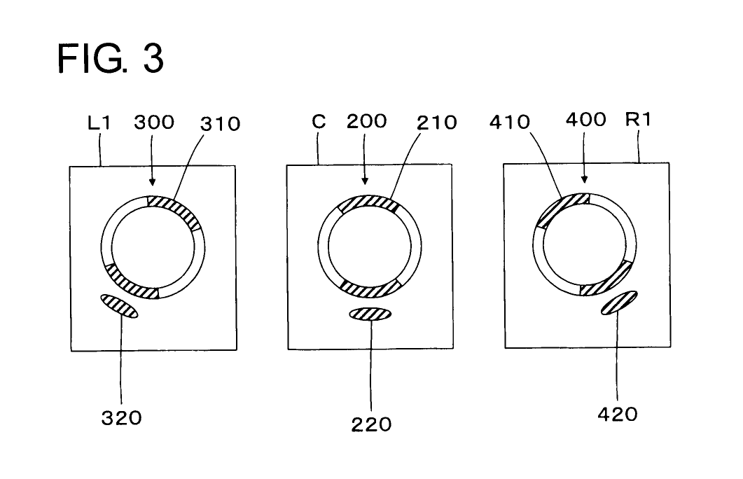

The following is a description of the relationship between the deflection angle of the ultrasound waves and the strength of the echo signals, with reference to FIG. 3. FIG. 3 is a schematic diagram of an ultrasound image. In each of the tomographic image data C, the tomographic image data L1, and the tomographic image data R1, images of blood vessels in the same short axis cross-section are shown. In the tomographic image data C, a blood-vessel image 200 is shown. In the tomographic image data L1, a blood-vessel image 300 is shown. In the tomographic image data R1, a blood-vessel image 400 is shown. The blood-vessel image 200, the blood-vessel image 300, and the blood-vessel image 400 are each images of the same short axis cross-section.

Even for a single structure of an organism, the strength of echo signals from a structure perpendicular to the transmission beams (ultrasound waves) becomes relatively high. Even though the position of the structure itself does not change, the distribution of the strength of the echo signals changes depending on the deflection angle of the transmission beams. At the same time, it appears an artifact on the axis of the transmission beams and the reception beams, at the position of the integral multiple of the distance to the ultrasound probe 1. This artifact is generated as a result of multiple reflections occurred at the structure of the organism and the surface of the ultrasound probe 1.

For example, in the tomographic image data C, within the blood-vessel image 200, a region 210 is a region that is perpendicular to the transmission beams. As a result, the strength of echo signals from the region 210 becomes relatively high. Because the tomographic image data C is an image based on ultrasound waves with a deflection angle of 0°, the region 210 corresponding to the upper and lower blood-vessel walls is visualized clearly. Moreover, on the axis of the transmission beams, a virtual image 220 appears.

In the tomographic image data L1, within the blood-vessel image 300, a region 310 is a region that is perpendicular to the transmission beams. As a result, the strength of echo signals from the region 310 becomes relatively high. Because the tomographic image data L1 is an image based on ultrasound waves deflected at the second deflection angle (left side in FIG. 2), the transmission beams are transmitted from the right side of the blood vessel. As a result, the region 310 that is tilted in accordance with the second deflection angle is visualized clearly. Moreover, a virtual image 320 also appears at a tilted position in accordance with the second deflection angle.

In the tomographic image data R1, within the blood-vessel image 400, a region 410 is a region that is perpendicular to the transmission beams. As a result, the strength of echo signals from the region 410 becomes relatively high. Because the tomographic image data R1 is an image based on ultrasound waves deflected at the third deflection angle (right side in FIG. 2), the transmission beams are transmitted from the left side of the blood vessel. As a result, the region 410 that is tilted in accordance with the third deflection angle is visualized clearly. Moreover, a virtual image 420 also appears at a tilted position in accordance with the third deflection angle.

As described above, when the deflection angle of transmission beams changes, the distribution of the strength of echo signals from a structure changes. Moreover, when the deflection angle of transmission beams changes, the position at which an artifact is generated and its strength also changes. In the present embodiment, changes in the strength distribution of echo signals from a structure caused by the deflection angle of transmission beams, as well as changes in the generated position of an artifact caused by the deflection angle of transmission beams, are patternized. For example, the strength distribution of echo signals from a structure and the generated position of an artifact are classified according to the deflection angle of the transmission beams. Furthermore, these classifications are predefined as patterns of the angular dependence (trends of the angular dependence) of the transmission beams.

### Patterns of Angular Dependence

The following shows an example of patterns of angular dependence.

Pattern SC: Echo signals from a structure easily reflecting transmission beams of the first deflection angle (deflection angle of) 0°.

Pattern SL: Echo signals from a structure easily reflecting transmission beams of the second deflection angle (deflected to the left side).

Pattern SR: Echo signals from a structure easily reflecting transmission beams of the third deflection angle (deflected to the right side).

Pattern AC: Artifact easily generated with transmission beams of the first deflection angle (deflection angle of 0°).

Pattern AL: Artifact easily generated with transmission beams of the second deflection angle (deflected to the left side).

Pattern AR: Artifact easily generated with transmission beams of the third deflection angle (deflected to the right side).

In the tomographic image data C shown in FIG. 3, the image of the region 210 corresponding to the blood-vessel wall corresponds to an image of the pattern SC. Moreover, in the tomographic image data C, the virtual image 220 corresponds to an artifact of the pattern AC. Moreover, in the tomographic image data L1 shown in FIG. 3, the image of the region 310 corresponding to the blood-vessel wall corresponds to an image of the pattern SL. Moreover, in the tomographic image data L1, the virtual image 320 corresponds to an artifact of the pattern AL. Moreover, in the tomographic image data R1 shown in FIG. 3, the image of the region 410 corresponding to the blood-vessel wall corresponds to an image of the pattern SR. Moreover, in the tomographic image data R1, the virtual image 420 corresponds to an artifact of the pattern AR.

### Calculator 5

The calculator 5 includes a difference-calculating part 51 and an angular-dependence determination part 52. The calculator 5 obtains the pattern of the angular dependence (trend of the angular dependence) among a plurality of ultrasound image data. The pattern of angular dependence is obtained based on multiple tomographic image data in which the deflection angle of ultrasound waves is different in each.

### Difference-Calculating Part 51

The difference-calculating part 51 obtains the differences between multiple tomographic image data in which the deflection angle of ultrasound waves is different in each. Moreover, the difference-calculating part 51 obtains the absolute value of each difference. If ultrasound waves are deflected at the first deflection angle, the second deflection angle, and the third deflection angle, the difference-calculating part 51 obtains the difference between each pair of the tomographic image data C (x, y), the tomographic image data L1 (x, y), and the tomographic image data R1 (x, y). Then, the difference-calculating part 51 obtains the absolute value of each obtained difference. Specifically, the difference-calculating part 51 obtains differences of pixel values such as luminance for each pixel (x, y), and also obtains the absolute values of these differences for each pixel (x, y). In one example, the difference-calculating part 51 obtains an absolute value CR (x, y) of the difference between the tomographic image data C (x, y) and the tomographic image data R1 (x, y) for each pixel (x, y). Moreover, the difference-calculating part 51 obtains an absolute value CL (x, y) of the difference between the tomographic image data C (x, y) and the tomographic image data L1 for each pixel (x, y). Moreover, the difference-calculating part 51 obtains an absolute value LR (x, y) of the difference between the tomographic image data L1 (x, y) and the tomographic image data R1 (x, y) for each pixel (x, y). The following are formulae for the absolute value CR (x, y), the absolute value CL (x, y), and the absolute value LR (x, y).

CR(x, y)=|C(x, y)−R1(x, y)|

CL(x, y)=|C(x, y)−L1(x, y)|

LR(x, y)=|L1(x, y)−R1(x, y)|

The difference-calculating part 51 outputs the absolute values CR (x, y), CL (x, y), and LR (x, y) of the differences obtained based on the above formulae to the angular-dependence determination part 52.

The above absolute values CR (x, y), CL (x, y), and LR (x, y) are used for determining the pattern of angular dependence (trend of angular dependence) of the tomographic image data on the deflection angle. The absolute values CR (x, y) and CL (x, y) are used for determining the direction of angular dependence. Specifically, using the first deflection angle as a standard, the absolute values CL (x, y) and CR (x, y) indicate in which direction the dependence is greater between the direction of the second deflection angle (leftward direction) and the direction of the third deflection angle (rightward direction). For example, if the absolute value CR (x, y) is greater than a preset threshold value, this indicates that the dependence on the direction of the third deflection angle (rightward direction) is greater. Moreover, the absolute value LR (x, y) indicates the size of the degree of angular dependence. As the difference between the tomographic image data L1 and the tomographic image data R1 becomes greater, the absolute value LR (x, y) becomes greater. As a result, it is possible to determine the size of the degree of angular dependence based on the absolute value LR (x, y). It should be noted that the absolute value CR (x, y) corresponds to one example of a first difference. Moreover, the absolute value CL (x, y) corresponds to one example of a second difference. Moreover, the absolute value LR (x, y) corresponds to one example of a third difference.

### Angular-Dependence Determination Part 52

The angular-dependence determination part 52 obtains the pattern of the angular dependence (trend of the angular dependence) of a plurality of ultrasound image data based on combinations of the absolute values of differences obtained by the difference-calculating part 51. Specifically, the angular-dependence determination part 52 determines the direction of the deflection angle in which echo signals have a relatively high strength. Alternatively, the angular-dependence determination part 52 determines the direction of the deflection angle in which the signal strength of an artifact is relatively high. For example, the threshold value for the absolute value CR (x, y) is defined as the threshold value Th1. Moreover, the threshold value for the absolute value CL (x, y) is defined as the threshold value Th2. Moreover, the threshold value for the absolute value LR (x, y) is defined as the threshold value Th3. These threshold values are standards for determining the pattern of angular dependence. These threshold values are preliminarily stored in a memory (not shown). Moreover, the operator may input the threshold values using the operation part 82.

The angular-dependence determination part 52 uses the threshold values Th1, Th2, Th3 to obtain an angular dependence pattern ADP (x, y) for each pixel (x, y). In one example, the angular dependence pattern ADP (x, y) is obtained according to seven conditions.

### First Condition

If CR (x, y)<Th1, CL (x, y)<Th2, and LR (x, y)<Th3, the angular dependence pattern ADP (x, y) is defined as “Pattern SC.” In other words, the angular-dependence determination part 52 determines that the strength of echo signals reflected from a structure due to transmission beams of the first deflection angle (deflection angle of 0°) is relatively high. If the absolute value CR (x, y) is less than the threshold value Th1 and the absolute value CL (x, y) is less than the threshold value Th2, it is inferred that the echo signals or artifacts are not dependent on the deflection angle. In other words, if the difference between the tomographic image data C (x, y) and the tomographic image data R1 (x, y) is relatively small and the difference between the tomographic image data C (x, y) and the tomographic image data L1 (x, y) is relatively small, it is inferred that the echo signals or artifacts are not dependent on the deflection angle. Moreover, if the absolute value LR (x, y) is less than the threshold value Th3, it is inferred that the difference between the tomographic image data L1 (x, y) and the tomographic image data R1 (x, y) is small and that the dependence on the direction of the second deflection angle (leftward direction) or the direction of the third deflection angle (rightward direction) is small. Consequently, if the absolute value CR (x, y), the absolute value CL (x, y), and the absolute value LR (x, y) meet the first condition, the angular-dependence determination part 52 defines the angular dependence pattern ADP (x, y) as “Pattern SC.”

### Second Condition

If CR (x, y)<Th1, CL (x, y)>Th2, and LR (x, y)>Th3, the angular dependence pattern ADP (x, y) is defined as “Pattern SL.” In other words, the angular-dependence determination part 52 determines that the strength of echo signals reflected from a structure due to transmission beams of the second deflection angle (deflected to the left side) is relatively high. If the absolute value CR (x, y) is less than the threshold value Th1 and the absolute value CL (x, y) is greater than the threshold value Th2, it is inferred that the echo signals or artifacts have angular dependence on the direction of the second deflection angle (leftward direction). In other words, if the difference between the tomographic image data C (x, y) and the tomographic image data R1 (x, y) is relatively small and the difference between the tomographic image data C (x, y) and the tomographic image data L1 (x, y) is relatively large, it is inferred that the echo signals or artifacts have angular dependence on the direction of the second deflection angle (leftward direction). Moreover, if the absolute value LR (x, y) is greater than the threshold value Th3, it is inferred that in the tomographic image data L1 (x, y) and the tomographic image data R1 (x, y), the difference in the strength of the echo signals from the structure is large. Because the signal strength of artifacts is relatively low, even if there is angular dependence, it is inferred that the difference between the tomographic image data L1 (x, y) and the tomographic image data R1 (x, y) becomes relatively small, and that the absolute value LR (x, y) becomes less than the threshold value Th3. On the other hand, because the strength of echo signals from the structure is relatively high, if there is angular dependence, it is inferred that the difference between the tomographic image data L1 (x, y) and the tomographic image data R1 (x, y) becomes relatively great, and that the absolute value LR (x, y) becomes greater than the threshold value Th3. Consequently, it is inferred that the difference between the tomographic image data C (x, y) and the tomographic image data L1 (x, y) is the difference in strength of the echo signals from the structure. Therefore, if the absolute value CR (x, y), the absolute value CL (x, y), and the absolute value LR (x, y) meet the second condition, the angular-dependence determination part 52 defines the angular dependence pattern ADP (x, y) as “Pattern SL.”

### Third Condition

If CR (x, y)>Th1, CL (x, y)<Th2, and LR (x, y)>Th3, the angular dependence pattern ADP (x, y) is defined as “Pattern SR.” In other words, the angular-dependence determination part 52 determines that the strength of echo signals reflected from a structure due to transmission beams of the third deflection angle (deflected to the right side) is relatively high. If the absolute value CR (x, y) is greater than the threshold value Th1 and the absolute value CL (x, y) is less than the threshold value Th2, it is inferred that the echo signals or artifacts have angular dependence on the direction of the third deflection angle (rightward direction). In other words, if the difference between the tomographic image data C (x, y) and the tomographic image data R1 (x, y) is relatively great and the difference between the tomographic image data C and the tomographic image data L1 is relatively small, it is inferred that the echo signals or artifacts have angular dependence on the direction of the third deflection angle (rightward direction). Moreover, as in the second condition, if the absolute value LR (x, y) is greater than the threshold value Th3, it is inferred that in the tomographic image data L1 (x, y) and the tomographic image data R1 (x, y), the difference in strength of the echo signals from the structure is great. Consequently, it is inferred that the difference between the tomographic image data C (x, y) and the tomographic image data R1 (x, y) is the difference in strength of the echo signals from the structure. Consequently, if the absolute value CR (x, y), the absolute value CL (x, y), and the absolute value LR (x, y) meet the third condition, the angular-dependence determination part 52 defines the angular dependence pattern ADP (x, y) as “Pattern SR.”

### Fourth Condition

If CR (x, y)>Th1, CL (x, y)>Th2, and LR (x, y)<Th3, the angular dependence pattern ADP (x, y) is defined as “Pattern AC.” In other words, the angular-dependence determination part 52 determines that artifacts are easily generated by transmission beams of the first deflection angle (deflection angle of 0°). If the absolute value CR (x, y) is greater than the threshold value Th1 and the absolute value CL (x, y) is greater than the threshold value Th2, it is inferred that the echo signals or artifacts have angular dependence on the second deflection angle (leftward direction) and the third deflection angle (rightward direction). In other words, if the difference between the tomographic image data C (x, y) and the tomographic image data R1 (x, y) is relatively great and the difference between the tomographic image data C (x, y) and the tomographic image data L1 (x, y) is relatively great, it is inferred that the echo signals or artifacts have angular dependence on the direction of the second deflection angle (leftward direction) or the direction of the third deflection angle (rightward direction). Moreover, if the absolute value LR (x, y) is less than the threshold value Th3, it is inferred that in the tomographic image data L1 (x, y) and the tomographic image data R1 (x, y), the difference in strength of echo signals from the structure is small. As described above, because the strength of the echo signals from the structure is relatively high, if there is angular dependence, it is inferred that the difference between the tomographic image data L1 (x, y) and the tomographic image data R1 (x, y) becomes relatively great and that the absolute value LR (x, y) becomes greater than the threshold value Th3. On the other hand, because the signal strength of artifacts is relatively low, even if there is angular dependence, it is inferred that the difference between the tomographic image data L1 (x, y) and the tomographic image data R1 (x, y) becomes relatively small and that the absolute value LR (x, y) becomes less than the threshold value Th3. Consequently, it is inferred that the difference between the tomographic image data C (x, y) and the tomographic image data R1 (x, y) is the difference in signal strength of the artifacts. Moreover, it is inferred that the difference between the tomographic image data C (x, y) and the tomographic image data L1 (x, y) is the difference in signal strength of the artifacts. Consequently, if the absolute value CR (x, y), the absolute value CL (x, y), and the absolute value LR (x, y) meet the fourth condition, the angular-dependence determination part 52 defines the angular dependence pattern ADP (x, y) as “Pattern AC.”

### Fifth Condition

If CR (x, y)<Th1, CL (x, y)>Th2, and LR (x, y)<Th3, the angular dependence pattern ADP (x, y) is defined as “Pattern AL.” In other words, the angular-dependence determination part 52 determines that artifacts are easily generated by transmission beams of the second deflection angle (deflected to the left side). As with the second condition, if the absolute value CR (x, y) is less than the threshold value Th1 and the absolute value CL (x, y) is greater than the threshold value Th2, it is inferred that the echo signals or artifacts have angular dependence on the direction of the second deflection angle (leftward direction). Moreover, as with the fourth condition, if the absolute value LR (x, y) is less than the threshold value Th3, it is inferred that in the tomographic image data L1 (x, y) and the tomographic image data R1 (x, y), the difference in strength of echo signals from the structure is small. Consequently, it is inferred that the difference between the tomographic image data C (x, y) and the tomographic image data L1 (x, y) is the difference in the signal strength of the artifacts. Consequently, if the absolute value CR (x, y), the absolute value CL (x, y), and the absolute value LR (x, y) meet the fifth condition, the angular-dependence determination part 52 defines the angular dependence pattern ADP (x, y) as “Pattern AL.”

### Sixth Condition

If CR (x, y)>Th1, CL (x, y)<Th2, and LR (x, y)<Th3, the angular dependence pattern ADP (x, y) is defined as “Pattern AR.” In other words, the angular-dependence determination part 52 determines that artifacts are easily generated by transmission beams of the third deflection angle (deflected to the right side). As with the third condition, if the absolute value CR (x, y) is greater than the threshold value Th1 and the absolute value CL (x, y) is less than the threshold value Th2, it is inferred that the echo signals or artifacts have angular dependence on the direction of the third deflection angle (rightward direction). Moreover, as with the fourth condition, if the absolute value LR (x, y) is less than the threshold value Th3, it is inferred that in the tomographic image data L1 (x, y) and the tomographic image data R1 (x, y), the difference in the strength of echo signals from the structure is small. Consequently, it is inferred that the difference between the tomographic image data C (x, y) and the tomographic image data R1 (x, y) is the difference in the signal strength of the artifacts. Consequently, if the absolute value CR (x, y), the absolute value CL (x, y), and the absolute value LR (x, y) meet the sixth condition, the angular-dependence determination part 52 defines the angular dependence pattern ADP (x, y) as “Pattern AR.”

### Seventh Condition

If the absolute value CR (x, y), the absolute value CL (x, y), and the absolute value LR (x, y) do not meet any of the above first to sixth conditions, the angular-dependence determination part 52 defines the angular dependence pattern ADP (x, y) as “Pattern 0.”

The angular-dependence determination part 52 outputs pattern information indicating the angular dependence pattern ADP (x, y) of each pixel (x, y) to the composition part 6.

### Composition Part 6

The composition part 6 generates composite image data by weighting each of multiple tomographic image data and composing them. In the present embodiment, the composition part 6 changes the weight of each pixel (x, y) of multiple tomographic image data according to the angular dependence pattern ADP (x, y) of each pixel (x, y). Based on this weighting, the composition part 6 composes multiple tomographic image data. For example, the composition part 6 changes the weights of the pixels (x, y) of each of the tomographic image data C (x, y), the tomographic image data L1 (x, y), and the tomographic image data R1 (x, y) according to the angular dependence pattern ADP (x, y). Based on this weighting, the composition part 6 composes the tomographic image data C (x, y), the tomographic image data L1 (x, y), and the tomographic image data R1 (x, y). In this way, the composition part 6 generates the composite image data TC (x, y). The following is a description of a method of composition according to the angular dependence pattern ADP (x, y).

### Case of Pattern SC

If the angular dependence pattern ADP (x, y) is “Pattern SC”, the composition part 6 obtains the composite image data TC (x, y) according to the following formula:

TC(x, y)={C(x, y)+L1(x, y)+R1(x, y)}/3

If the strength of echo signals reflected from the structure due to transmission beams of the first deflection angle (deflection angle of 0°) is relatively high, the composition part 6 averages all of the tomographic image data. As a result of this averaging, the composition part 6 generates the composite image data TC (x, y). That is, the composition part 6 gives equal weight to each of the tomographic image data C (x, y), the tomographic image L1 (x, y), and the tomographic image data R1 (x, y) and adds them. As a result of this addition, the composition part 6 generates the composite image data TC (x, y). In other words, the composition part 6 selects all of the tomographic image data from among the tomographic image data C (x, y), the tomographic image data L1 (x, y), and the tomographic image data R1 (x, y), and averages all of the tomographic image data. As a result of this averaging, the composition part 6 generates the composite image data TC (x, y).

### Case of Pattern SL If the angular dependence pattern ADP (x, y) is “Pattern SL”, the composition part 6 obtains the composite image data TC (x, y) according to the following formula:

TC(x, y)={C(x, y)+L1(x, y)}/2

If the strength of echo signals reflected from the structure due to transmission beams of the second deflection angle (deflected to the left side) is relatively high, the composition part 6 averages the tomographic image data C (x, y) and the tomographic image data L1 (x, y). As a result of this averaging, the composition part 6 generates the composite image data TC (x, y). In other words, the composition part 6 assigns a weight of “0.5” to the tomographic image data C (x, y) and the tomographic image data L1 (x, y), and assigns a weight of “0” to the tomographic image data R1 (x, y). Based on this weighting, the composition part 6 adds the tomographic image data C (x, y), the tomographic image data L1 (x, y), and the tomographic image data R1 (x, y). As a result of this addition, the composition part 6 generates the composite image data TC (x, y). In other words, the composition part 6 selects the tomographic image data C (x, y) and the tomographic image data L1 (x, y) from among the tomographic image data C (x, y), the tomographic image data L1 (x, y), and the tomographic image data R1 (x, y), and averages the tomographic image data C (x, y) and the tomographic image data L1 (x, y). As a result of this averaging, the composite image data TC (x, y) is generated. If the angular dependence pattern ADP (x, y) is Pattern SL, the two tomographic image data, from among the three tomographic image data, in which the sensitivity of the echo signals is high are averaged. In this case, the two tomographic image data are the tomographic image data C and the tomographic image data L1. Because the tomographic image data R1, in which the sensitivity is low, is not used for averaging, it is possible to reduce decreases in sensitivity caused by averaging compared to cases in which all three tomographic image data are averaged.

### Case of Pattern SR

If the angular dependence pattern ADP (x, y) is “Pattern SR,” the composition part 6 obtains the composite image data TC (x, y) according to the following formula:

TC(x, y)={C(x, y)+R1(x, y)}/2

If the strength of echo signals reflected from the structure due to transmission beams of the third deflection angle (deflected to the right side) is relatively high, the composition part 6 averages the tomographic image data C (x, y) and the tomographic image data R1 (x, y). As a result of this averaging, the composition part 6 generates the composite image data TC (x, y). In other words, the composition part 6 assigns a weight of “0.5” to the tomographic image data C (x, y) and the tomographic image data R1 (x, y), and assigns a weight of “0” to the tomographic image data L1 (x, y). Based on this weighting, the composition part 6 adds the tomographic image data C (x, y), the tomographic image data L1 (x, y), and the tomographic image data R1 (x, y). As a result of this addition, the composition part 6 generates the composite image data TC (x, y). In other words, the composition part 6 selects the tomographic image data C (x, y), and the tomographic image data R1 (x, y) from among the tomographic image data C (x, y). the tomographic image data L1 (x, y), and the tomographic image data R1 (x, y), and averages the tomographic image data C (x, y) and the tomographic image data R1 (x, y). As a result of this averaging, the composition part 6 generates the composite image data TC (x, y). If the angular dependence pattern ADP (x, y) is Pattern SR, the two tomographic image data, from among the three tomographic image data, in which the sensitivity of the echo signals is high are averaged. In this case, the two tomographic image data are the tomographic image data C and the tomographic image data R1. Because the tomographic image data L1, in which the sensitivity is low, is not used for averaging, it is possible to reduce decreases in sensitivity caused by averaging compared to cases in which all three tomographic image data are averaged.

### Case of Pattern AC

If the angular dependence pattern ADP (x, y) is “Pattern AC”, the composition part 6 obtains the composite image data TC (x, y) according to the following formula:

TC(x, y)={L1(x, y)+R1(x, y)}/2

If artifacts are easily generated due to transmission beams of the first deflection angle (deflection angle of 0°), the composition part 6 averages the tomographic image data L1 (x, y) and the tomographic image data R1 (x, y). As a result of this averaging, the composition part 6 generates the composite image data TC (x, y). In other words, the composition part 6 assigns a weight of “0” to the tomographic image data C (x, y) and assigns a weight of “0.5” to the tomographic image data L1 (x, y) and the tomographic image data R1 (x, y). Based on this weighting, the composition part 6 adds the tomographic image data C (x, y), the tomographic image data L1 (x, y), and the tomographic image data R1 (x, y). As a result of this addition, the composition part 6 generates the composite image data TC (x, y). In other words, the composition part 6 selects the tomographic image data L1 (x, y) and the tomographic image data R1 (x, y) from among the tomographic image data C (x, y), the tomographic image data L1 (x, y), and the tomographic image data R1 (x, y), and averages the tomographic image data L1 (x, y) and the tomographic image data R1 (x, y). As a result of this averaging, the composition part 6 generates the composite image data TC. If the angular dependence pattern ADP (x, y) is Pattern AC, the two tomographic image data, from among the three tomographic image data, in which the sensitivity of artifacts is low are averaged. In this case, the two tomographic image data are the tomographic image data L1 and the tomographic image data R1. Because the tomographic image data C, in which the sensitivity of artifacts is high, is not used for averaging, it is possible to restrain increases of artifacts caused by averaging compared to cases in which all three tomographic image data are averaged.

### Case of Pattern AL

If the angular dependence pattern ADP (x, y) is “Pattern AL”, the composition part 6 obtains the composite image data TC (x, y) according to the following formula:

TC(x, y)={C(x, y)+R1(x, y)}/2

If artifacts are easily generated due to transmission beams of the second deflection angle (deflected to the left side), the composition part 6 averages the tomographic image data C (x, y) and the tomographic image data R1 (x, y). As a result of this averaging, the composition part 6 generates the composite image data TC (x, y). In other words, a weight of “0” is assigned to the tomographic image data L1 (x, y), and a weight of “0.5” is assigned to the tomographic image data C (x, y) and the tomographic image data R1 (x, y). Based on this weighting, the composition part 6 adds the tomographic image data C (x, y), the tomographic image data L1 (x, y), and the tomographic image data R1 (x, y). As a result of this addition, the composition part 6 generates the composite image data TC (x, y). In other words, the composition part 6 selects the tomographic image data C (x, y) and the tomographic image data R1 (x, y) from among the tomographic image data C (x, y), the tomographic image data L1 (x, y), and the tomographic image data R1 (x, y), and averages the tomographic image data C (x, y) and the tomographic image data R1 (x, y). As a result of this averaging, the composition part 6 generates the composite image data TC (x, y). If the angular dependence pattern ADP (x, y) is Pattern AL, the two tomographic image data, from among the three tomographic image data, in which the sensitivity of artifacts is low are averaged. In this case, the two tomographic image data are the tomographic image data C and the tomographic image data R1. Because the tomographic image data L1, in which the sensitivity of artifacts is high, is not used for averaging, it is possible to restrain increases of artifacts caused by averaging compared to cases in which all three tomographic image data are averaged.

### Case of Pattern AR

If the angular dependence pattern ADP (x, y) is “Pattern AR”, the composition part 6 obtains the composite image data TC (x, y) according to the following formula:

TC(x, y)={C(x, y)+L1(x, y)}/2

If artifacts are easily generated due to transmission beams of the third deflection angle (deflected to the right side), the composition part 6 averages the tomographic image data C (x, y) and the tomographic image data L1 (x, y). As a result of this averaging, the composition part 6 generates the composite image data TC (x, y). In other words, the composition part 6 assigns a weight of “0” to the tomographic image data R1 (x, y), and assigns a weight of “0.5” to the tomographic image data C (x, y) and the tomographic image data L1 (x, y). Based on this weighting, the composition part 6 adds the tomographic image data C (x, y), the tomographic image data L1 (x, y), and the tomographic image data R1 (x, y). As a result of this addition, the composition part 6 generates the composite image data TC (x, y). In other words, the composition part 6 selects the tomographic image data C (x, y) and the tomographic image data L1 (x, y) from among the tomographic image data C (x, y), the tomographic image data L1(x, y), and the tomographic image data R1 (x, y), and averages the tomographic image data C (x, y) and the tomographic image data L1 (x, y). As a result of this averaging, the composition part 6 generates the composite image data TC. If the angular dependence pattern ADP (x, y) is Pattern AR, the two tomographic image data, from among the three tomographic image data, in which the sensitivity of artifacts is low are averaged. In this case, the two tomographic image data are the tomographic image data C and the tomographic image data L1. Because the tomographic image data R1, in which the sensitivity of artifacts is high, is not used for averaging, it is possible to restrain increases of artifacts caused by averaging compared to cases in which all three tomographic image data are averaged.

### Case of Pattern 0

If the angular dependence pattern ADP (x, y) is “Pattern 0,” the composition part 6 obtains the composite image data TC (x, y) according to the following formula:

TC(x, y)={C(x, y)+L1(x, y)+R1(x, y)}/3

By performing the above composition for each pixel (x, y), the composition part 6 generates the composite image data TC (x, y) for each pixel (x, y). The composition part 6 outputs the composite image data TC (x, y) to the display controller 7.

As described above, if the echo signals from a structure have angular dependence, it is possible to prevent decreases in sensitivity caused by averaging tomographic image data with high sensitivity from among the three tomographic image data. Moreover, if the artifacts have angular dependence, it is possible to restrain increases of artifacts caused by averaging tomographic image data with low sensitivity from among the three tomographic image data. In other words, for echo signals from a structure, tomographic image data with high sensitivity are selected, and for artifacts, tomographic image data with low sensitivity are selected. Based on these factors, it becomes possible to generate images with high sensitivity for structures and unnoticeable artifacts. As a result, compared to images based on artifacts, the visibility of images based on biological signals (echo signals from structures) improves.

### Modified Example of Composition Method

The following is a description of a modified example of the composition method. The composition part 6 may generate the composite image data TC (x, y) by using the maximum or minimum pixel values from among multiple tomographic image data.

For example, if the angular dependence pattern ADP (x, y) is any one of “Pattern SC,” “Pattern SL,” or “Pattern SR,” the composition part 6 obtains the composite image data TC (x, y) according to the following formula:

TC(x, y)=Max{C(x, y), L1(x, y), R1(x, y)}

If the echo signals from a structure have angular dependence, the composition part 6 generates the composite image data TC (x, y) using the maximum pixel value from among the three tomographic image data. In other words, the composition part 6 selects the maximum pixel value from among the tomographic image data C (x, y), the tomographic image data L1 (x, y), and the tomographic image data R1 (x, y) to generate the composite image data TC (x, y).

In other words, the composition part 6 assigns a weight of “1” to the maximum pixel value, and assigns a weight of “0” to pixel values other than the maximum value, and performs weighted addition of the tomographic image data C (x, y), the tomographic image data L1 (x, y), and the tomographic image data R1 (x, y) to generate the composite image data TC (x, y). If the echo signals from a structure have angular dependence, by using the maximum pixel value from among the three tomographic image data, it becomes possible to select tomographic image data in which the strength of echo signals from the structure is relatively high.

Moreover, if the angular dependence pattern ADP (x, y) is any of “Pattern AC,” “Pattern AL,” or “Pattern AR,” the composition part 6 obtains the composite image data TC (x, y) according to the following formula:

TC(x, y)=Min{C(x, y), L1(x, y), R1(x, y)}

If an artifact has angular dependence, the composition part 6 generates the composite image data TC (x, y) using the minimum pixel value from among the three tomographic image data. In other words, the composition part 6 selects the minimum pixel value from among the tomographic image data C (x, y), the tomographic image data L1 (x, y), and the tomographic image data R1 (x, y) to generate the composite image data TC (x, y). In other words, the composition part 6 assigns a weight of “0” to the minimum pixel value, and assigns a weight of “0” to pixel values other than the minimum value, and performs weighted addition of the tomographic image data C (x, y), the tomographic image data L1 (x, y), and the tomographic image data R1 (x, y) to generate the composite image data TC (x, y). If an artifact has angular dependence, by using the minimum pixel value from among the three tomographic image data, it becomes possible to select tomographic image data in which the signal strength of the artifact is relatively low.

If the angular dependence pattern ADP (x, y) is “Pattern 0,” the composition part 6 obtains the composite image data TC (x, y) according to the following formula:

TC(x, y)={C(x, y)+L1(x, y)+R1(x, y)}/3

In other words, the composition part 6 generates the composite image data TC (x, y) by averaging all of the tomographic image data. p According to a composition method of the modified example described above, if the echo signals from a structure have angular dependence, tomographic image data with the highest sensitivity is selected, and if an artifact has angular dependence, tomographic image data with the lowest sensitivity is selected. Based on these factors, it becomes possible to generate images with high sensitivity for structures and unnoticeable artifacts. As a result, compared to images based on artifacts, the visibility of images based on biological signals (echo signals from structures) improves.

### Display Controller 7

The display controller 7 receives the composite image data TC (x, y) from the composition part 6. Based on the received composite image data TC (x, y), the display controller 7 displays a composite image on a display 81.

### User Interface (UI) 8

The user interface (UI) 8 includes the display 81 and the operation part 82. The display 81 is configured by a display device such as a CRT or a liquid crystal display. The operation part 82 is configured by an input device such as a keyboard or a mouse.

### Controller 9

The controller 9 controls the actions of each part of the ultrasound diagnosis apparatus. For example, the controller 9 controls the transmission and reception of ultrasound waves by the transceiver 2.

It should be noted that a composition process for three tomographic image data has been described, but three or more tomographic image data may be composed. Moreover, the ultrasound diagnosis apparatus of the present embodiment may compose multiple volume data, and may compose multiple three-dimensional image data.

### Modified Example of Compound Scan

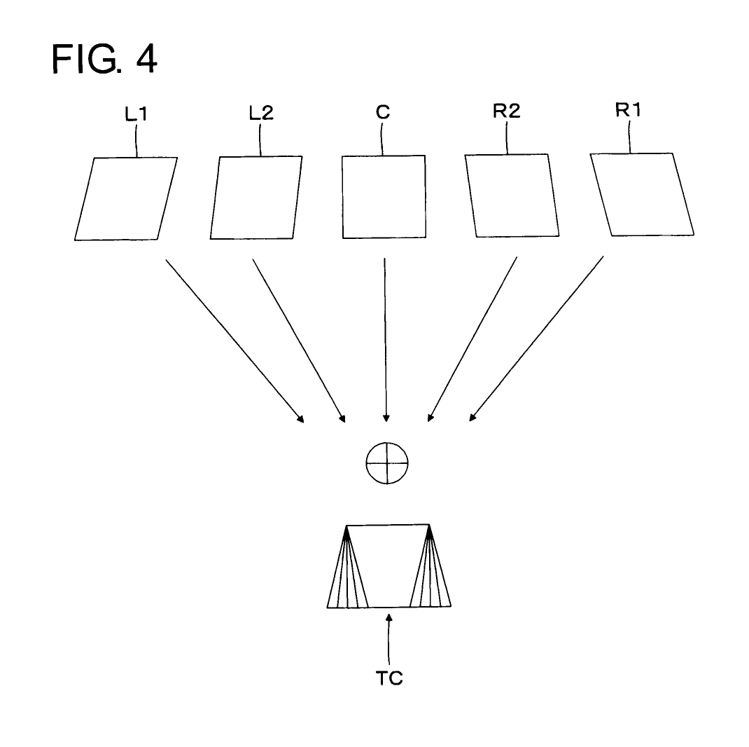

The following is a description of a modified example of a compound scan, with reference to FIG. 4. FIG. 4 is a diagram showing the concept of a scan. In this modified example, the ultrasound probe 1 deflects ultrasound waves at, for example, five different deflection angles and transmits and receives ultrasound waves. Moreover, based on received echo signals, the ultrasound diagnosis apparatus generates five tomographic image data that have different deflection angles. Under the control of the controller 9, the transceiver 2 deflects ultrasound waves at a first deflection angle, a second deflection angle, a third deflection angle, a fourth deflection angle, and a fifth deflection angle and transmits and receives ultrasound waves. As described above, the first deflection angle is 0°. The second deflection angle is the angle of the most leftward deflection in FIG. 4. The third deflection angle is the angle of the most rightward deflection in FIG. 4. The fourth deflection angle is an angle between the first deflection angle and the second deflection angle. The fifth deflection angle is an angle between the first deflection angle and the third deflection angle. The first deflection angle, the second deflection angle, the third deflection angle, the fourth deflection angle, and the fifth deflection angle may be input by the operator using the operation part 82.

The image-generating part 4 generates tomographic image data C that is based on ultrasound waves deflected at the first deflection angle. Moreover, it generates tomographic image data L1 that is based on ultrasound waves deflected at the second deflection angle. Moreover, it generates tomographic image data R1 that is based on ultrasound waves deflected at the third deflection angle. Moreover, it generates tomographic image data L2 that is based on ultrasonic waves deflected at the fourth deflection angle. Moreover, it generates the tomographic image data R2 that is based on the ultrasound waves deflected at the fifth deflection angle. FIG. 4 shows each tomographic image data. The tomographic image data C shown in FIG. 4 is image data in which ultrasound waves have been deflected at the first deflection angle (deflection angle of 0°). The tomographic image data L1 is image data based on ultrasound waves deflected at the second deflection angle (most leftward in FIG. 4). The tomographic image data R1 is image data based on ultrasound waves deflected at the third deflection angle (most rightward in FIG. 4). The tomographic image data L2 is image data based on ultrasound waves deflected at the fourth deflection angle (left side in FIG. 4). The tomographic image data R2 is image data based on ultrasound waves deflected at the fifth deflection angle (right side in FIG. 4). The tomographic image data C, the tomographic image data L1, the tomographic image data R1, the tomographic image data L2, and the tomographic image data R2 are composed by the composition part 6. As a result of this composition, the composite image data TC is generated.

Even in a case in which ultrasound waves are transmitted and received by deflecting ultrasound waves at five deflection angles as described above, the calculator 5 uses tomographic image data with great differences in the deflection angle. In other words, the calculator 5 uses the tomographic image data C (x, y) in which the deflection angle is 0°, the tomographic image data L1 (x, y) in which the ultrasound waves have been deflected most leftward, and the tomographic image data R1 (x, y) in which the ultrasound waves have been deflected most rightward to obtain the angular dependence pattern ADP (x, y) for each pixel (x, y). This is because by using tomographic image data with great differences in the deflection angle, the trend of the angular dependence becomes clear.

As described above, the composition part 6 changes the weights of each tomographic image data according to the angular dependence pattern ADP (x, y). Based on this, the composition part 6 weighs each tomographic image data and composes them. In the present modified example, the composition part 6 changes the weights of the pixels (x, y) of each of the tomographic image data C (x, y), the tomographic image data L1 (x, y), the tomographic image data L2 (x, y), the tomographic image data R1 (x, y), and the tomographic image data R2 (x, y) according to the angular dependence pattern ADP (x, y). Based on this weighting, the composition part 6 composes the tomographic image data C (x, y), the tomographic image data L1 (x, y), the tomographic image data L2 (x, y), the tomographic image data R1 (x, y), and the tomographic image data R2 (x, y). In this way, the composition part 6 generates the composite image data TC (x, y). By performing the above composition for each pixel (x, y), the composition part 6 generates the composite image data TC (x, y) for each pixel (x, y). The composition part 6 outputs the composite image data TC (x, y) to the display controller 7.

Furthermore, even in cases in which ultrasound waves are deflected at seven or more deflection angles to transmit and receive ultrasound waves, the ultrasound diagnosis apparatus may obtain the angular dependence pattern ADP (x, y) by using tomographic image data with great differences in deflection angle.

The respective functions of the image-generating part 4, the calculator 5, the composition part 6, and the display controller 7 may be executed by programs. In one example, the image-generating part 4, the calculator 5, the composition part 6, and the display controller 7 may each be configured by a processing device (not shown) and a memory device (not shown). The processing device may be configured by a CPU, a GPU, or an ASIC, etc. The memory device may be configured by a ROM, a RAM, or an HDD, etc. The memory device stores an image-generating program, a calculation program, a composition program, and a display processing program. The image-generating program executes the functions of the image-generating part 4. The composition program executes the functions of the composition part 6. The calculation program executes the functions of the calculator 5. The display processing program executes the functions of the display controller 7. Moreover, the calculation program includes a difference-calculating program and an angular-dependence determination program. The difference-calculating program executes the functions of the difference-calculating part 51. The angular-dependence program executes the functions of the angular-dependence determination part 52. The processing device such as a CPU executes the functions of each part by executing each program stored in the memory.

### Actions

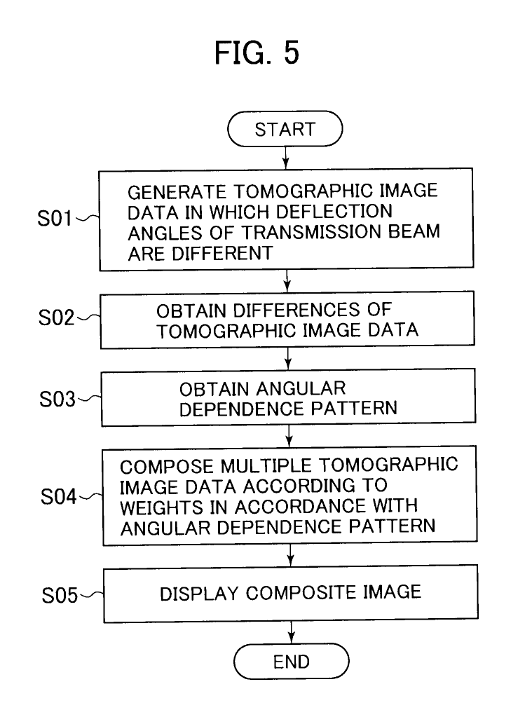

The following is a description of a series of actions performed by the ultrasound diagnosis apparatus according to the first embodiment, with reference to FIG. 5. FIG. 5 is a flowchart showing a series of actions performed by the ultrasound diagnosis apparatus according to the first embodiment.

### Step S01

First, under the control of the controller 9, the transceiver 2 changes the deflection angle and transmits and receives ultrasound waves. For example, the transceiver 2 deflects ultrasound waves at the first deflection angle (deflection angle of 0°), the second deflection angle, and the third deflection angle and transmits and receives ultrasound waves. The image-generating part 4 generates the tomographic image data C in which the ultrasound waves have been deflected at the first deflection angle, as shown in FIG. 2, for example. Moreover, it generates the tomographic image data L1 in which the ultrasound waves have been deflected at the second deflection angle. Moreover, it generates the tomographic image data R1 in which the ultrasound waves have been deflected at the third deflection angle. The image-generating part 4 outputs the tomographic image data to the calculator 5 and the composition part 6.

### Step S02

The difference-calculating part 51 obtains the differences between multiple tomographic image data in which the deflection angles of the ultrasound waves are different. Then, the difference-calculating part 51 obtains the absolute value of each difference that has been obtained. For example, the difference-calculating part 51 obtains the differences between the tomographic image data C (x, y), the tomographic image data L1 (x, y), and the tomographic image data R1 (x, y) for each pixel (x, y). Then, the difference-calculating part 51 obtains the absolute values CR (x, y), CL (x, y), and LR (x, y) of each difference that has been obtained.

### Step S03

Based on combinations of the absolute values of the differences obtained by the difference-calculating part 51, the angular-dependence determination part 52 obtains the angular dependence pattern ADP (x, y) for each pixel (x, y). The angular-dependence determination part 52 outputs pattern information indicating the angular dependence pattern ADP (x, y) to the composition part 6.

### Step S04

The composition part 6 changes the weight of each pixel (x, y) of multiple tomographic image data according to the angular dependence pattern ADP (x, y) of each pixel (x, y). Based on this weighting, the composition part 6 composes multiple tomographic image data. For example, the composition part 6 changes the weights of the pixels (x, y) of each of the tomographic image data C (x, y), the tomographic image data L1 (x, y), and the tomographic image data R1 (x, y) according to the angular dependence pattern ADP (x, y). Based on this weighting, the composition part 6 composes the tomographic image data C (x, y), the tomographic image data L1 (x, y), and the tomographic image data R1 (x, y) to generate the composite image data TC (x, y) for each pixel (x, y). The composition part 6 outputs the composite image data TC (x, y) to the display controller 7.

### Step S05

The display controller 7 displays a composite image based on the composite image data TC (x, y) on the display 81.

As described above, according to the ultrasound diagnosis apparatus according to the first embodiment, if echo signals from a structure have angular dependence, by averaging tomographic image data with high sensitivity from among multiple tomographic image data, it becomes possible to prevent decreases in sensitivity caused by averaging. Moreover, if an artifact has angular dependence, by averaging tomographic image data with low sensitivity from among multiple tomographic image data, it becomes possible to restrain increases of artifacts caused by averaging. Based on these factors, it becomes possible to generate images with high sensitivity for structures and unnoticeable artifacts. As a result, compared to images based on artifacts, the visibility of images based on biological signals (echo signals from structures) improves.

### Second Embodiment

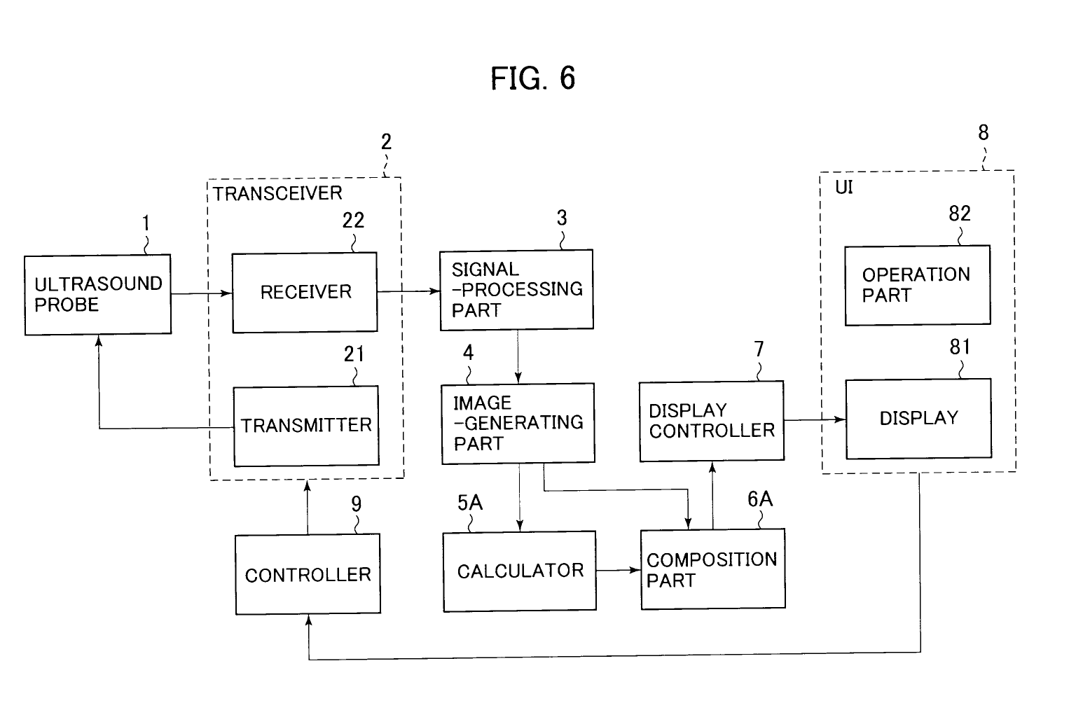

The following is a description of an ultrasound diagnosis apparatus according to a second embodiment, with reference to FIG. 6. FIG. 6 is a block diagram of an ultrasound diagnosis apparatus according to the second embodiment. The ultrasound diagnosis apparatus according to the second embodiment includes a calculator 5A and a composition part 6A instead of the calculator 5 and the composition part 6 of the first embodiment. Configurations other than the calculator 5A and the composition part 6A are identical to those of the ultrasound diagnosis apparatus according to the first embodiment, and descriptions thereof are therefore omitted. As one example, a case is described in which, as in the first embodiment, ultrasound waves are deflected at a first deflection angle, a second deflection angle, and a third deflection angle, and the tomographic image data C (x, y), the tomographic image data L1 (x, y), and the tomographic image data R1 (x, y) are generated.

### Calculator 5A

Based on multiple tomographic image data that have different deflection angles of the ultrasound waves, the calculator 5A obtains angular dependence information ADI (x, y) indicating the trend of the angular dependence for each pixel (x, y). As shown in the following formula, the absolute value of the difference in the pixel values of the tomographic image data L1 (x, y) and the tomographic image data R1 (x, y) is defined as the angular dependence information ADI (x, y).

ADI(x, y)=|L1(x, y)−R1(x, y)|

This shows that the greater the angular dependence information ADI (x, y) is, the greater the degree of angular dependence becomes. The calculator 5A outputs the angular dependence information ADI (x, y) to the composition part 6A.

### Composition Part 6A

The composition part 6A averages multiple tomographic image data that have different deflection angles of the ultrasound waves. As a result of this averaging, the composition part 6A generates composite image data. For example, the composition part 6A averages the tomographic image data C (x, y), the tomographic image data L1 (x, y), and the tomographic image data R1 (x, y). As a result of this averaging, the composition part 6A generates the composite image data TC (x, y).

Moreover, by composing the angular dependence information ADI (x, y) onto the composite image data TC (x, y), the composition part 6A generates superimposed image data TI (x, y). For example, the composition part 6A composes the angular dependence information ADI (x, y) onto any one of the R signal (Red signal), G signal (Green signal), and B signal (Blue signal) of the composite image data TC (x, y). The operator may, for example, use the operation part 82 to designate the signal onto which the angular dependence information ADI (x, y) is composed. As one example, if the angular dependence information ADI (x, y) is composed onto the B signal, the composition part 6A generates the superimposed image data TI (x, y) (R, G, B) according to the following formulae:

R signal of TI(x, y)=TC(x, y)

G signal of TI(x, y)=TC(x, y)

B signal of TI(x, y)=TC(x, y)+ADI (x, y)

The composition part 6A outputs the superimposed image data TI (x, y) to the display controller 7. The display controller 7 displays a superimposed image based on the superimposed image data TI (x, y) on the display 81.

As described above, the composition part 6A composes the angular dependence information ADI (x, y) onto the composite image data TC (x, y). As a result of this composition, the composition part 6A causes parts where the angular dependence is great (i.e., the angular dependence information ADI is great) to be displayed with the color of the composed component emphasized. For example, if the angular dependence information ADI (x, y) is composed onto the B signal, the part where the angular dependence information ADI (x, y) has been composed is displayed with a tincture of blue. If the angular dependence is great, that is, if the angular dependence information ADI is great, the blue is displayed in a bolder shade according to the size of the angular dependence information ADI. As a result, it is easier for the operator to discriminate between the structure of an organism and an artifact.

It should be noted that, although a composition process involving three tomographic image data has been described, as in the first embodiment, three or more tomographic image data may be composed. Moreover, multiple volume data may be composed, and multiple three-dimensional image data may be composed.

Moreover, the first embodiment and the second embodiment may be combined. For example, the composition part 6A may compose the angular dependence information ADI (x, y) onto the composite image data TC (x, y) generated by the composition part 6 according to the first embodiment.

The respective functions of the calculator 5A and the composition part 6A may be executed by programs. As one example, the calculator 5A and the composition part 6A may be respectively configured by a processing device (not shown) and a memory device (not shown). The processing device may be configured by a CPU, a GPU, or an ASIC, etc. The memory device may be configured by a ROM, a RAM, or an HDD, etc. The memory device stores a calculation program and a composition program. The calculation program executes the functions of the calculator 5A. The composition program executes the functions of the composition part 6A. The processing device, which is, for example, a CPU, executes the functions of each part by executing each program stored in the memory.

### Actions

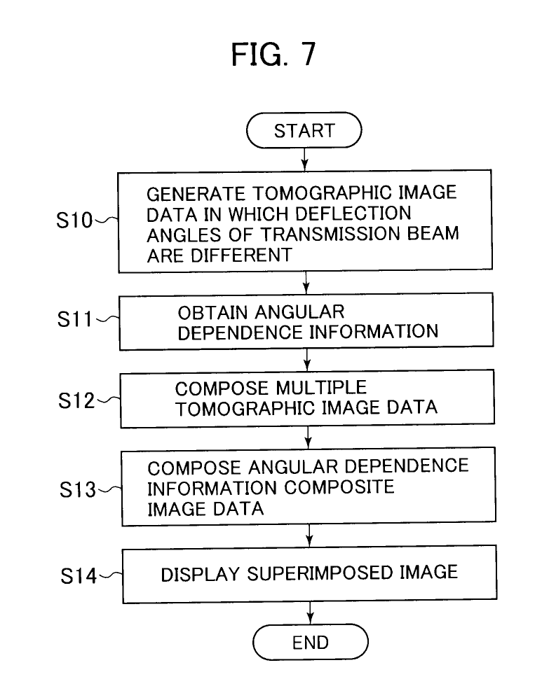

The following is a description of a series of actions performed by the ultrasound diagnosis apparatus according to the second embodiment, with reference to FIG. 7. FIG. 7 is a flowchart showing a series of actions performed by the ultrasound diagnosis apparatus according to the second embodiment.

### Step S10

First, under the control of the controller 9, the transceiver 2 changes the deflection angle and transmits and receives ultrasound waves. For example, the transceiver 2 deflects ultrasound waves at the first deflection angle (deflection angle of 0°), the second deflection angle, and the third deflection angle and transmits and receives ultrasound waves. As shown in FIG. 2, the image-generating part 4 generates the tomographic image data C in which the ultrasound waves have been deflected at the first deflection angle. Moreover, it generates the tomographic image data L1 in which the ultrasound waves have been deflected at the second deflection angle. Moreover, it generates the tomographic image data R1 in which the ultrasound waves have been deflected at the third deflection angle. The image-generating part 4 outputs the tomographic image data to the calculator 5A and the composition part 6A.

### Step S11

The calculator 5A obtains the angular dependence information ADI (x, y) for each pixel (x, y) based on the multiple tomographic image data that have different deflection angles for the ultrasound waves. For example, the calculator 5A defines the absolute value of the difference in pixel values between the tomographic image data L1 (x, y) and the tomographic image data R1 (x, y) as the angular dependence information ADI (x, y). The calculator 5A outputs the angular dependence information ADI (x, y) to the composition part 6A.

### Step S12

The composition part 6A averages the multiple tomographic image data that each different deflection angles for the ultrasound waves. As a result of this averaging, the composition part 6A generates composite image data. For example, the composition part 6A averages the tomographic image data C (x, y), the tomographic image data L1 (x, y), and the tomographic image data R1 (x, y). As a result of this averaging, the composition part 6 generates the composite image data TC (x, y).

### Step S13

The composition part 6A composes the angular dependence information ADI (x, y) onto the composite image data TC (x, y). As a result of this composition, the composition part 6A generates the superimposed image data TI (x, y). For example, the composition part 6A composes the angular dependence information ADI (x, y) onto the B signal of the composite image data TC (x, y). The composition part 6A outputs the superimposed image data TI (x, y) to the display controller 7.

### Step S14

The display controller 7 displays a superimposed image based on the superimposed image data TI (x, y) on the display 81.

As described above, according to the ultrasound diagnosis apparatus according to the second embodiment, the angular dependence information ADI (x, y) is composed onto the composite image data TC (x, y). As a result of this composition, parts where the angular dependence is great (i.e., the angular dependence information ADI is great) are displayed in the color of the composed component (e.g., blue) emphasized. If the angular dependence is great (i.e., if the angular dependence information ADI is great), the blue color, for example, is displayed in a bolder shade, thereby making it easier for the operator to discriminate between the structure of an organism and an artifact.

### Third Embodiment

The following is a description of an ultrasound diagnosis apparatus according to a third embodiment. As in the first embodiment, the ultrasound diagnosis apparatus according to the third embodiment also obtains the angular dependence pattern ADP (x, y) of multiple images (each pixel in the images) obtained by a compound scan. Moreover, it is also similar in that each image is assigned weights. However, unlike the first embodiment, the third embodiment changes the composition method according to the imaging area or the trend. In other words, in accordance with the part, etc. shown in an image, a setting is made for either a composition method in which the pixels of each weighted image are averaged, or a composition method using the maximum or minimum values of each weighted image.

For example, for an image showing a part where random noise such as speckles is easily occurred, the implementation of smoothing with the composition method of averaging weighted images is more effective for eliminating the noise. In contrast, for an image showing a structure such as the cross-section of a blood vessel, it is effective to generate an image with high sensitivity through the composition method using the maximum value of each weighted image. Moreover, for such an image, it is effective to generate an image in which artifacts are unnoticeable through the composition method using the minimum value of each image.

### Composition-Method Setting Screen

In the third embodiment, a memory (not shown) in the ultrasound diagnosis apparatus stores image data of a setting screen for the composition method. The setting screen for the composition method is not shown in the diagrams. The setting screen for the composition method includes a display field for setting the “Imaging area or Trend.” Imaging areas in the display field include, for example, a blood vessel or a liver, etc. For trends, the likelihood of noise occurrence is displayed in steps, for example. When the operator uses the operation part 82 to select an imaging area or a trend from among the items displayed on the display field, the selected information is output to the controller 9.

In the memory (not shown), composition methods corresponding to the imaging area or trend are stored in advance. The controller 9 reads out from the memory a composition method corresponding to the information of, for example, the imaging area selected in the composition method setting screen.

It should be noted that this is not limited to a configuration in which the controller 9 reads out a corresponding composition method based on the angular dependence pattern. Other examples include configurations such as the following. When an angular dependence pattern is obtained, the angular dependence pattern is displayed. At the same time, at least one tomographic image is displayed. At this point, the operator is able to refer to the displayed tomographic image and the obtained angular dependence pattern. In this example, the composition method setting screen is provided with a display field that displays multiple types of composition methods. When the operator uses the operation part 82 to select a composition method from among the items displayed in this display field, the information is sent to the controller 9.

The controller 9 outputs the composition method set as described above to the composition part 6.

### Composition Method

The following is a description of the generation of the composite image data TC (x, y) by the composition part 6 of the third embodiment.

### Case of Pattern SC

If the angular dependence pattern ADP (x, y) is “Pattern SC” and “Imaging area is a blood vessel” is selected, the composition part 6 obtains the composite image data TC (x, y) according to the following formula:

TC(x, y)=Max {C(x, y), L1(x, y), R1(x, y)}

In other words, the same weighting is assigned to each tomographic image data and the maximum pixel value is selected to generate the composite image data TC (x, y). It should be noted that the definition of Pattern SC is the same as in the first embodiment.

Furthermore, if the angular dependence pattern ADP (x, y) is “Pattern SC” and “low degree of noise occurrence” is selected as the image trend, the composition part 6 obtains the composite image data TC (x, y) in the same manner as in the above case in which “Imaging area is a blood vessel” is selected.

If the angular dependence pattern ADP (x, y) is “Pattern SC” and “Imaging area is a liver” is selected, the composition part 6 obtains the composite image data TC (x, y) according to the following formula:

TC(x, y)={C(x, y)+L1(x, y)+R1(x, y)}/3

In other words, the tomographic image data are assigned the same weight and added. In this way, the composition part 6 generates the composite image data TC (x, y).

If the angular dependence pattern ADP (x, y) is “Pattern SC” and “high degree of noise occurrence” is selected as the image trend, the composition part 6 obtains the composite image data TC (x, y) in the same manner as in the above case in which “Imaging area is a liver” is selected.

### Case of Pattern SL

If the angular dependence pattern ADP (x, y) is “Pattern SL” and “Imaging area is a blood vessel” is selected, the composition part 6 obtains the composite image data TC (x, y) according to the following formula:

TC(x, y)=Max{C(x, y), L1(x, y), R1(x, y)}

Furthermore, the same applies even if the angular dependence pattern ADP (x, y) is “Pattern SL” and “low degree of noise occurrence” is selected as the image trend.

If angular dependence pattern ADP (x, y) is “Pattern SL” and “Imaging area is a liver” is selected, the composition part 6 obtains the composite image data TC (x, y) according to the following formula:

TC(x, y)={C(x, y)+L1(x, y)}/2

Furthermore, the same applies even if the angular dependence pattern ADP (x, y) is “Pattern SL” and “high degree of noise occurrence” is selected as the image trend.

### Case of Pattern SR

If the angular dependence pattern ADP (x, y) is “Pattern SR” and “Imaging area is a blood vessel” is selected, the composition part 6 obtains the composite image data TC (x, y) according to the following formula:

TC(x, y)=Max{C(x, y), L1(x, y), R1(x, y)}

Furthermore, the same applies even if angular dependence pattern ADP (x, y) is “Pattern SR” and “low degree of noise occurrence” is selected as the image trend.

If the angular dependence pattern ADP (x, y) is “Pattern SR” and “Imaging area is a liver” is selected, the composition part 6 obtains the composite image data TC (x, y) according to the following formula:

TC(x, y)={C(x, y)+R1(x, y)}/2

Furthermore, the same applies even if angular dependence pattern ADP (x, y) is “Pattern SR” and “high degree of noise occurrence” is selected as the image trend.

### Case of Pattern AC

If the angular dependence pattern ADP (x, y) is “Pattern AC” and “Imaging area is a blood vessel” is selected, the composition part 6 obtains the composite image data TC (x, y) according to the following formula:

TC(x, y)=Min{C(x, y), L1(x, y), R1(x, y)}

The composition part 6 uses the minimum pixel value from among the multiple tomographic image data to generate the composite image data TC (x, y). Furthermore, the same applies even if the angular dependence pattern ADP (x, y) is “Pattern AC” and “low degree of noise occurrence” is selected as the image trend.

If the angular dependence pattern ADP (x, y) is “Pattern AC” and “Imaging area is a liver” is selected, the composition part 6 obtains the composite image data TC (x, y) according to the following formula:

TC(x, y)={L1(x, y)+R1(x, y)}/2

The composition part 6 assigns a weight of “0” to the tomographic image data C (x, y) and a weight of “0.5” to the tomographic image data L1 (x, y) and the tomographic image data R1 (x, y). Based on this weighting, the composition part 6 adds the tomographic image data C (x, y), the tomographic image data L1 (x, y), and the tomographic image data R1 (x, y). As a result of this addition, the composition part 6 generates the composite image data TC (x, y). Furthermore, the same applies even if the angular dependence pattern ADP (x, y) is “Pattern AC” and “high degree of noise occurrence” is selected as the image trend.

### Case of Pattern AL

If the angular dependence pattern ADP (x, y) is “Pattern AL” and “Imaging area is a blood vessel” is selected, the composition part 6 obtains the composite image data TC (x, y) according to the following formula:

TC(x, y)=Min{C(x, y), L1(x, y), R1(x, y)}

The composition part 6 uses the minimum pixel value from among the multiple tomographic image data to generate the composite image data TC (x, y). Furthermore, the same applies even if the angular dependence pattern ADP (x, y) is “Pattern AL” and “low degree of noise occurrence” is selected as the image trend.

If the angular dependence pattern ADP (x, y) is “Pattern AL” and “Imaging area is a liver” is selected, the composition part 6 obtains the composite image data TC (x, y) according to the following formula:

TC(x, y)={C(x, y)+R1(x, y)}/2

The composition part 6 assigns a weight of “0” to the tomographic image data L1 (x, y) and a weight of “0.5” to the tomographic image data C (x, y) and the tomographic image data R1 (x, y). Based on this weighting, the composition part 6 adds the multiple tomographic image data. As a result of this addition, the composition part 6 generates the composite image data TC (x, y). Furthermore, the same applies even if the angular dependence pattern ADP (x, y) is “Pattern AL” and “high degree of noise occurrence” is selected.

### Case of Pattern AR

If the angular dependence pattern ADP (x, y) is “Pattern AR” and “Imaging area is a blood vessel” is selected, the composition part 6 obtains the composite image data TC (x, y) according to the following formula:

TC(x, y)=Min{C(x, y), L1(x, y), R1(x, y)}

The composition part 6 uses the minimum pixel value from among the multiple tomographic image data to generate the composite image data TC (x, y). Furthermore, the same applies even if the angular dependence pattern ADP (x, y) is “Pattern AR” and “low degree of noise occurrence” is selected as the image trend.

If the angular dependence pattern ADP (x, y) is “Pattern AR” and “Imaging area is a liver” is selected, the composition part 6 obtains the composite image data TC (x, y) according to the following formula:

TC(x, y)={C(x, y)+L1(x, y)}/2

The composition part 6 assigns a weight of “0” to the tomographic image data R1 (x, y) and a weight of “0.5” to the tomographic image data C (x, y) and the tomographic image data L1 (x, y). Based on this weighting, the composition part 6 adds the multiple tomographic image data. As a result of this addition, the composition part 6 generates the composite image data TC (x, y). Furthermore, the same applies even if the angular dependence pattern ADP (x, y) is “Pattern AR” and “high degree of noise occurrence” is selected as the image trend.

### Case of Pattern 0

If the angular dependence pattern ADP (x, y) is “Pattern 0,” regardless of the information selected on the composition method selection screen, the composition part 6 obtains the composite image data TC (x, y) according to the following formula:

TC(x, y)={C(x, y)+L1(x, y)+R1(x, y)}/3

By performing the above composition for each pixel (x, y), the composition part 6 generates the composite image data TC (x, y) for each pixel (x, y). The composition part 6 outputs the composite image data TC (x, y) to the display controller 7.

### Threshold-Value Setting Screen

In the third embodiment, the memory (not shown) of the ultrasound diagnosis apparatus stores a setting screen for threshold values for pixel values, etc. that define the angular dependence pattern. In other words, the threshold values Th1, Th2, Th3 used for determining whether the first to sixth conditions described above are met are input via the threshold-value setting screen. This threshold-value setting screen is not shown in the diagrams. As described above, the threshold value Th1 is the threshold value for the absolute value CR (x, y). The threshold value Th2 is the threshold value for the absolute value CL (x, y). The threshold value Th3 is the threshold value for the absolute value LR (x, y).

The threshold-value setting screen is provided with an input field for the threshold value Th1, an input field for the threshold value Th2, and an input field for the threshold value Th3. When the operator uses the operation part 82 to input a threshold value into any of the input fields, the input threshold value and the type thereof (Th1, Th2, or Th3) is output to the controller 9.

It should be noted that the threshold-value setting screen is not necessarily of a configuration including input fields for each of the threshold value Th1, the threshold value Th2, and the threshold value Th3. For example, a display field in which combinations of threshold values may be selected for each “imaging area” or “image trend” may be provided. The “imaging area” and “image trend” are the same as those in the description of the composition method setting screen. As one example, in the display field of the threshold-value setting screen, combinations of the threshold value Th1, the threshold value Th2, and the threshold value Th3 for cases of “Imaging area is a blood vessel” are selectable. Moreover, as another example, combinations of the threshold value Th1, the threshold value Th2, and the threshold value Th3 for cases in which the image trend shows a “high degree of noise occurrence” are selectable.

When the operator uses the operation part 82 to select an item in the display field, the selected combination of threshold values is output to the controller 9.

The controller 9 sends the input or selected combination of threshold values to the angular-dependence determination part 52. The angular-dependence determination part 52 uses the threshold values Th1, Th2, Th3 to obtain the angular dependence pattern ADP (x, y) for each pixel (x, y).

Furthermore, the third embodiment may be applied even in cases in which ultrasound waves are deflected at five deflection angles to transmit and receive ultrasound waves, or cases in which ultrasound waves are deflected at seven or more deflection angles to transmit and receive ultrasound waves. Moreover, the third embodiment may be applied to an ultrasound diagnosis apparatus in combination with the second embodiment. Moreover, the third embodiment has been described using only examples in which the imaging area is a “blood vessel” or a “liver,” but of course, in the various setting screens, other imaging areas may be selected, etc. Moreover, the “degree of noise occurrence” has been described as the image trend, but the image trend may be displayed using other expressions. Moreover, in the third embodiment, it is sufficient if any one of the change in the composition method and the change in the threshold value may be performed, and it is not always necessary to perform both processes.

As described above, according to the ultrasound diagnosis apparatus according to the third embodiment, the composition method is changed according to the imaging area or the image trend. In other words, if the echo signals from a structure have angular dependence, it is possible to switch between performing averaging and using the maximum value according to the imaging area or the image trend. Moreover, if an artifact has angular dependence, it is possible to switch between performing averaging and using the minimum value according to the imaging area or the image trend.

That is, depending on the situation, harmonization between suppression of both decreases in sensitivity caused by averaging and increases in artifacts are suppressed, and the elimination of noises is achieved. Based on these factors, it becomes possible to generate images with high sensitivity for structures and unnoticeable artifacts. As a result, compared to images based on artifacts, the visibility of images based on biological signals (echo signals from structures) improves.

Furthermore, in combination with the second embodiment, the angular dependence information ADI (x, y) is composed onto the composite image data TC (x, y). As a result of this composition, parts in which the angular dependence is great (i.e., the angular dependence information ADI is great) are displayed with the color of the composed component (e.g., blue) emphasized. If the angular dependence is great (i.e., if the angular dependence information ADI is great), the blue color (for example) is displayed in a bolder shade, thereby making it easier for the operator to discriminate between the structure of an organism and an artifact.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

### EXPLANATION OF THE SYMBOLS

1: Ultrasound probe
2: Transceiver
3: Signal-processing part
4: Image-generating part
5, 5A: Calculator
6, 6A: Composition part
7: Display controller
8: User interface (UI)
9: Controller
51: Difference-calculating part
52: Angular-dependence determination part

1. An ultrasound diagnosis apparatus comprising:
an image-capturing part that deflects ultrasound waves at a plurality of different deflection angles, transmits ultrasound waves to a subject, receives echo signals from said subject, and generates a plurality of ultrasound image data in which said deflection angles of said ultrasound waves are different for each;
a calculator that, based on said plurality of ultrasound image data, obtains the trend of the angular dependence of said plurality of ultrasound image data on said deflection angles; and
a composition part that weighs each of said plurality of ultrasound image data in accordance with the trend of said angular dependence, and composes said plurality of ultrasound image data.

2. The ultrasound diagnosis apparatus according to claim 1, wherein
said calculator obtains the differences between said plurality of ultrasound image data, and obtains the trend of said angular dependence based on a combination of said differences.

3. The ultrasound diagnosis apparatus according to claim 2, wherein
said calculator obtains, based on the combination of said differences, the direction of deflection angles in which either the strength of echo signals from a structure in said subject or the signal strength of an artifact becomes relatively high as the trend of said angular dependence, and
said composition part composes, from among said plurality of ultrasound image data, ultrasound image data at deflection angles in which the strength of said echo signals from said structure becomes relatively high.

4. The ultrasound diagnosis apparatus according to claim 3, wherein
said image-capturing part deflects said ultrasound waves at a first deflection angle to generate a first ultrasound image data at said first deflection angle, deflects said ultrasound waves at a second deflection angle different from said first deflection angle to generate a second ultrasound image data at said second deflection angle, and deflects said ultrasound waves at a third deflection angle on the opposite side of said second deflection angle in relation to said first deflection angle to generate a third ultrasound image data at said third deflection angle,
said calculator obtains a first difference between said first ultrasound image data and said second ultrasound image data, a second difference between said first ultrasound image data and said third ultrasound image data, and a third difference between said second ultrasound image data and said third ultrasound image data, and based on said first difference and said second difference, obtains the direction of deflection angles in which either the strength of said echo signals from said structure or the signal strength of said artifact becomes relatively high, and based on said third difference, determines which of either the strength of said echo signals from said structure or the signal strength of said artifact becomes relatively high, and
said composition part composes, if the strength of said echo signals from said structure becomes relatively high, ultrasound image data in which the strength of said echo signals from said structure becomes relatively high from among said first ultrasound image data, said second ultrasound image data, and said third ultrasound image data, and if the signal strength of said artifact becomes relatively high, composes ultrasound image data in which the signal strength of said artifact becomes relatively low from among said first ultrasound image data, said second ultrasound image data, and said third ultrasound image data.

5. The ultrasound diagnosis apparatus according to claim 4, wherein
said calculator
compares a preset first threshold value with said first difference and compares a preset second threshold value with said second difference, thereby determining whether the strength of echo signals from a structure inside said subject or the signal strength of an artifact is dependent on either of said second deflection angle or said third deflection angle, and
compares a preset third threshold value with said third difference, thereby determining which of either the strength of said echo signals from said structure or the signal strength of said artifact becomes relatively high.

6. The ultrasound diagnosis apparatus according to claim 5, further comprising:
a user interface that is used for setting said first threshold value, said second threshold value, and said third threshold value, wherein
said calculator, based on said first threshold value, said second threshold value, and said third threshold value set in said user interface, compares the first threshold value with said first difference, compares the second threshold value with said second difference, and compares the third threshold value with said third difference.

7. The ultrasound diagnosis apparatus according to claim 4, further comprising:
a user interface that is used for specifying an imaging area of said ultrasound image; and
a memory that associates and stores a plurality of composition methods by said composition part with the direction of said deflection angle, a determination result of which of either the strength of said echo signals from said structure or the signal strength of said artifact is relatively high, and said specified imaging area,
wherein said composition part, based on the direction of said deflection angle obtained by said calculator and said determination result as well as said imaging area, selects one of said composition methods, and composes ultrasound image data based on this composition method.

8. An ultrasound diagnosis apparatus comprising:
an image-capturing part that deflects ultrasound waves at a plurality of different deflection angles, transmits ultrasound waves to a subject, receives echo signals from said subject, and generates a plurality of ultrasound image data in which said deflection angles of said ultrasound waves are different for each;
a calculator that, based on said plurality of ultrasound image data, obtains the trend of the angular dependence of said plurality of ultrasound image data on said deflection angles;
a composition part that generates composite image data by composing said plurality of ultrasound image data, and composes information indicating the trend of said angular dependence on said composite image data.

9. The ultrasound diagnosis apparatus according to claim 8, wherein
said calculator obtains the differences between said plurality of ultrasound image data, and obtains the trend of said angular dependence based on said differences.

10. The ultrasound diagnosis apparatus according to claim 8, wherein
said composition part composes on said composite image data information indicating the trend of said angular dependence as a prescribed color.

11. The ultrasound diagnosis apparatus according to claim 9, wherein
said composition part composes on said composite image data information indicating the trend of said angular dependence as a prescribed color.
